SEQUENCE LISTING (3 sequences)

SEQ ID NO: 1 - DNA, 335 bp, synthetic construct
  source 1..335: mol_type = other DNA
aatgatacggcgaccaccgagatctacacnnnnnnnnacactctttccctacacgacgctcttccgatctsgcttccatttcaggtgtcgtgaagcggccgcacgcgtccgnnnnnnnnnnnnnnnnnnnngccaccatggtcgacnnnnnnnnnnnnnnnnnnnncggtagcggatccgtgagcaagggcgaggagctgttcaccggggtggtgcccatcctggtcgagctggacggcgacgtaaacggccacaagttcagcgtgtccagatcggaagagcacacgtctgaactccagtcacnnnnnnnnatctcgtatgccgtcttctgcttg

SEQ ID NO: 2 - DNA, 86 bp, synthetic construct
  source 1..86: mol_type = other DNA
caagcagaagacggcatacgagatnnnnnnnngtgactggagttcagacgtgtgctcttccgatctggacacgctgaacttgtggc

SEQ ID NO: 3 - DNA, 70 bp, synthetic construct
  source 1..70: mol_type = other DNA
aatgatacggcgaccaccgagatctacacnnnnnnnnacactctttccctacacgacgctcttccgatct

(19) United States
(12) Patent Application Publication
(10) Pub. No.: US 2025/0236905 A1
(43) Pub. Date: Jul. 24, 2025

(54) CARPOOL: A High Throughput in Vitro and in Vivo Screening Platform

(71) Applicant: New York University, New York, NY (US)

(72) Inventors: Erik Sulman, White Plains, NY (US); Zeyan Zhang, New York, NY (US); Yingwen Ding, New York, NY (US)

(21) Appl. No.: 18/831,439

(22) Filed: Jan. 24, 2025

Related U.S. Application Data
(60) Provisional application No. 63/624,442, filed on Jan. 24, 2024.

(51) Int. Cl. C12Q 1/6809; C12Q 1/6869; G01N 33/50

(52) U.S. Cl. CPC C12Q 1/6809; C12Q 1/6869; G01N 33/5011; G01N 33/5044; C12Q 2600/136; G01N 2500/10

(57) ABSTRACT

Provided are compositions and methods for analyzing groups of barcoded cells by screening the effects of test agents. The methods can be used both in vitro and in vivo and facilitate high-throughput, concurrent identification of the effects of multiple test agents on different groups of cells.

### CROSS-REFERENCE TO RELATED APPLICATIONS

This application claims the benefit of priority to U.S. provisional patent application No. 63/624,442, filed Jan. 24, 2024, the entire disclosure of which is incorporated herein by reference.

### SEQUENCE LISTING

The instant application contains a Sequence Listing, which has been submitted electronically in XML format and is hereby incorporated by reference in its entirety. Said XML copy, created on Jan. 24, 2025, is named 058636.00782.xml, and is 3,986 bytes in size.

### BACKGROUND

Currently, pre-clinical evaluation of most therapies has been tested to a handful of cell models, due to the cost and time required to conduct experiments are large number of models using many conditions. Many diseases, such as cancer, exhibit considerable genomic heterogeneity, making it challenging to generalize from the use of only a few cell lines. Indeed, the high failure rate for drug developing of cancer therapeutics make in large part be due to the failure to analyse sufficient numbers of models to reflect the heterogeneity of the disease. Thus, there is an unmet need to maximize the coverage of tumor heterogeneity by evaluating the response of oncology therapeutics using at large number of models, including the patient-derived models. There is also a need to develop biomarker-guided therapeutics based on the molecular characteristics of responding cell lines and increases the success rate of oncology therapeutic clinical trials. The present disclosure is pertinent to this need.

### BRIEF SUMMARY

The present disclosure provides compositions and methods for analyzing groups of barcoded cells during screening the effects of test agents. The compositions and methods can be used in vitro and in vivo. The described approaches include sequentially: providing a series of groups of test cells; barcoding each test cell such that all the test cells in a single group in the series of groups contains the same barcode, and wherein the barcodes are different in each group of test cells; pooling the groups of test cells to obtain a pooled combination of the groups of test cells; exposing the pooled combination to a test agent and maintaining the pooled combination in the presence of a test agent for a period of time; adding a known number of control cells to the pooled combination, wherein each of the control cells comprises a control barcode and wherein the control barcode is different from any of the barcodes in the test cells, and wherein control cells are not affected by the test agent; and sequencing the barcodes of the test cells and the control barcode of the control cells to determine a difference between the amount of control cells and the amount of test cells to thereby determine an effect of the test agent on each group of the test cells.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1: Testing fluoxetine (FLUO) sensitivities of patient-derived glioblastoma spheroid cell lines (GSCs) in vitro using in vitro CARPOOL (Panel a) a heatmap showing fluoxetine (FLUO) sensitivities of a pool of 20 GSCs obtained by in vitro CARPOOL approach. EGFR expression and TCGA GBM subtypes of the GSCs were annotated to explore their potential correlation with FLUO sensitivities. (Panel b) Sigmoidal curve fitting of results from in vitro CARPOOL experiment to calculate the IC50 values of FLUO to the 20 GSCs. Blue and red lines highlighted the most sensitive and resistant GSCs, respectively. (Panel c) Sigmoldal curve fitting of results from conventional ATP-based cell viability assay (CellTiter-Glo) to calculate the IC50 values of FLUO to the 20 GSCs. Blue and red lines highlighted the most sensitive and resistant GSCs, respectively. (Panel d) Pearson correlation of IC50 values obtained from CARPOOL and conventional approaches.

FIG. 2: Testing temozolomide (TMZ) sensitivities of GSCs in vitro using in vitro CARPOOL (Panel a) a heatmap showing TMZ sensitivities of a pool of 20 GSCs obtained by in vitro CARPOOL approach. Methylation status (MGMT status) and Cancer Genome Atlas Glioblastoma Multiforme (TCGA-GBM) glioblastoma multiform (GBM) subtypes of the GSCs, as well as TMZ sensitivities classified by single line conventional approach, were annotated to explore their potential correlation with TMZ sensitivities. (Panel b) Sigmoidal curve fitting of results from in vitro CARPOOL experiment to calculate the IC50 values of TMZ to the 20 GSCs. Blue and red lines highlighted the most sensitive and resistant GSCs, respectively. (Panel c) Sigmoidal curve fitting of results from conventional ATP-based cell viability assay (CeilTiter-Glo) to calculate the IC50 values of TMZ to the 20 GSCs. Blue and red lines highlighted the most sensitive and resistant GSCs, respectively.

FIG. 3. Using in vitro CARPOOL for combination treatments. The heatmap showing in vitro CARPOOL results of combing FLUO and TMZ treatments of 20 GSCs. Epidermal growth factor receptor (EGFR) expression, EGFR mutation status, IDH status, G-CIMP class, MGMT status and TCGA GBM subtypes of the GSCs were annotated to explore their potential correlation with FLUO sensitivities.

FIG. 4: Using in vitro CARPOOL for drug screening. In vitro CARPOOL was applied for an in vitro drug screening to profiled sensitivity of 728 compounds over 23 GSCs. (Panel a) a heatmap showing sensitivities, determined as the area under the cure (AUC) of the sigmoidal fitting curves, of each of the 728 screening compounds across 23 GSCs. GSCs source patient gender, MGMT status and TCGA GBM subtypes of the GSCs were annotated to explore their potential correlation with drug sensitivities. The horizontal boxplot on the left of the heatmap showing the distribution of the sensitivities of GSCs to each of the compound. (Panel b) A density plot showing the AUCs of drugs classified by their previously involved in oncology study or not. The dashed line showing the median AUC of each class of compounds. Panel (c) Pie charts showing the proportions of compounds from each of the two class (Oncology compounds vs other compounds) compounds that are broadly active, selective, ineffective from in vitro CARPOOL screening. The 728-compound library was obtained from MedChem Express, Catalog number HY-L028.

FIG. 5: An example of in vitro CARPOOL drug screening results that identified biomarker of drug sensitivities. (Panel a) Volcano plot showing the relationship of 728 compounds and TP53 mutational status. The x-axis showing the effect size between TP53 wild-type and mutant GSCs, and the y-axis showing −log 10 formatted Wicoxon p-value of the comparison. (Panel b) A boxplot showing the distribution of RG7112, a MDM2 inhibitor, sensitivities in TP53 wild-type and mutant GSCs.

FIG. 6: Using CARPOOL in vivo. (Panel a) Experimental design to compare the proliferate kinetics when growth in vivo and in vitro; and to evaluate GSCs' sensitivity to temozolomide (TMZ) using in vivo CARPOOL (Panel b) IVIS images showing tumor burden at 21 days post implantation of mice from each group.

FIG. 7: Increased total barcodes over time of the mice after implantation quantified using quantitative polymerase chain reaction (qPCR) by in vivo CARPOOL Normal brain without barcoded GSC implantation was used as negative control.

FIG. 8: In vivo GSCs quantification using qPCR by in vivo CARPOOL Bar-charts showing relative quantity (upper plots) or absolute quantity (lower plots) of representative rapidly proliferating GSC17 Panel (a), moderately proliferating GSC11 (Panel b), and slowly proliferating GS5-22 (Panel c).

FIG. 9: Responses detectable in a wide range of proportions by in vivo CARPOOL Bar-charts showing the responses of GSCs with various proportions (maximum difference tested was ˜4000-fold) due to differently proliferating speeds.

FIG. 10: Monitoring growth of pooled GSCs in vitro and in vivo by CARPOOL (Panel a) Stacked area plots showing the relative proportions of GSCs overtime while growth in vitro or in vivo. (Panel b) Pearson correlations of GSCs growth in vitro and in vivo at the indicated time points.

FIG. 11: In vivo sensitivities of GSCs to TMZ treatment determined by in vivo CARPOOL (Panels a and b) Boxplots showing in vivo TMZ responses (relative cell viability) of mice treated with one-week (Panel a) or two-week (Panel b) TMZ. The number on the plot showing Wilcoxon test p values. (Panel c) A heatmap showing the average in vivo TMZ responses (relative cell viability) of mice treated with one-week or two-week. (Panel d) Pearson correlation of average in vivo TMZ responses between one-week and two-week TMZ treatments.

FIG. 12: Validating the TMZ sensitivity from in vivo CARPOOL by mouse survival analysis. Two in vivo CARPOOL TMZ sensitive GSCs, two in vivo CARPOOL TMZ medium GSCs and two in vivo CARPOOL TMZ resistant GSCs were used to perform mouse survival analysis. Mice implanted with each of the validated GSC were treated with TMZ (50 mg/kg/day) or vehicle (0.5% methyl cellulose) for three cycles (5 days in week 1 (Monday to Friday) followed by three weeks of drug holidays). Humane endpoints were recorded for Kaplan-Meier (KM) survival analysis. The KM survival curves showing the results.

FIG. 13. Workflow of CARPOOL for high throughput therapeutic screening in vitro and in vivo with patient-derived glioblastoma spheroid cultures (GSCs). (Panel a). Preparation of barcoded cells for therapeutic screening. Each patient-derived GSC was tagged by a unique DNA barcode, and the barcoded GSCs were pooled for treatment as a pool. (Panel b). Preparation of spike-in cells to generate standard curve for absolute cell counting. Additional cell lines (e.g., U87, but other types of cells can be used) were labelled with different barcodes, and a series of known counts of different barcode were mixed as spike-in cells. (Panel c) Therapeutic screening workflow in vitro and in vivo. For in vitro, pooled GSCs were seeded in 96-well plates for treating. Non-viable cells were removed by FBS induced live cell attachment. Spike-in cells were loaded into each well before cell lysis. The barcodes counts were accessed by amplicon NGS or amplicon qPCR, and spike-in cells counts was used to obtain absolute cell counts for each GSCs. For in vivo, the pooled GSCs were intracranially implanted followed by treatments. The quarter brains with GSCs implantation were harvested, and spike-in cells were added before genome DNA (gDNA) extraction. The barcodes counts were accessed by amplicon NGS or amplicon qPCR, and spike-in cells counts was used to obtain absolute cell counts for each GSCs.

FIG. 14. Representative standard curve showing normalized sequencing reads and cell number by comparison of spike-in control and test cells.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein directly or by reference. Each RNA sequence includes its DNA equivalent, and each DNA sequence includes its RNA equivalent. Complementary and anti-parallel polynucleotide sequences are included. Sequences of from 80.00%-99.99% identical to any sequence (amino adds and nucleotide sequences) of this disclosure are included.

The disclosure includes all polynucleotide and all amino acid sequences that are identified herein by way of a database entry. Such sequences are incorporated herein as they exist in the database on the filing date of this application or patent.

The disclosure includes each feature illustrated by the accompanying figures, each component of each feature individually, and all combinations thereof.

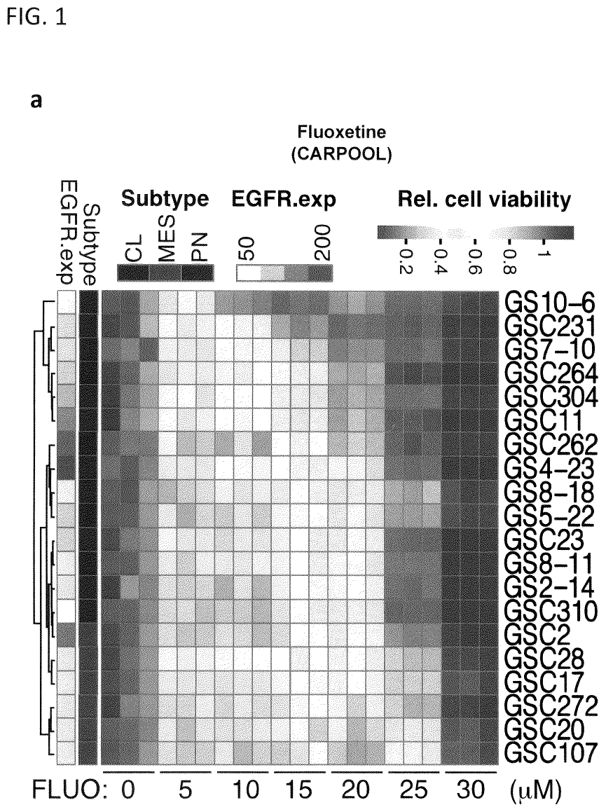

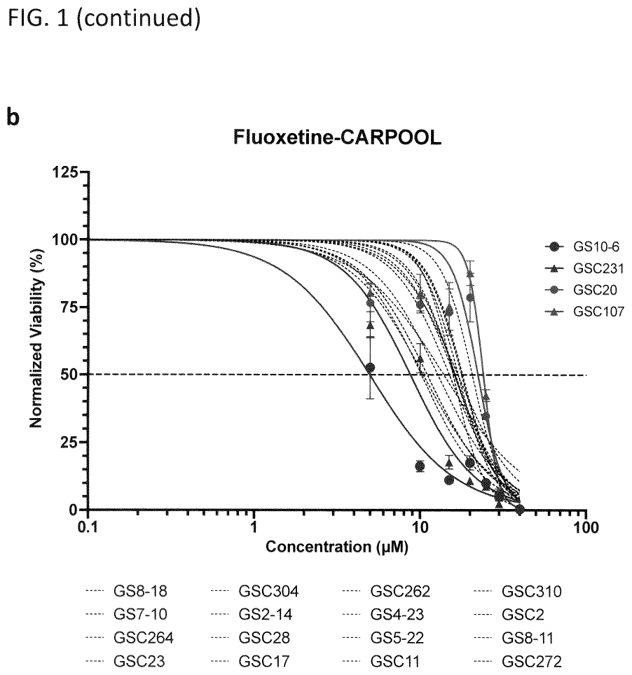

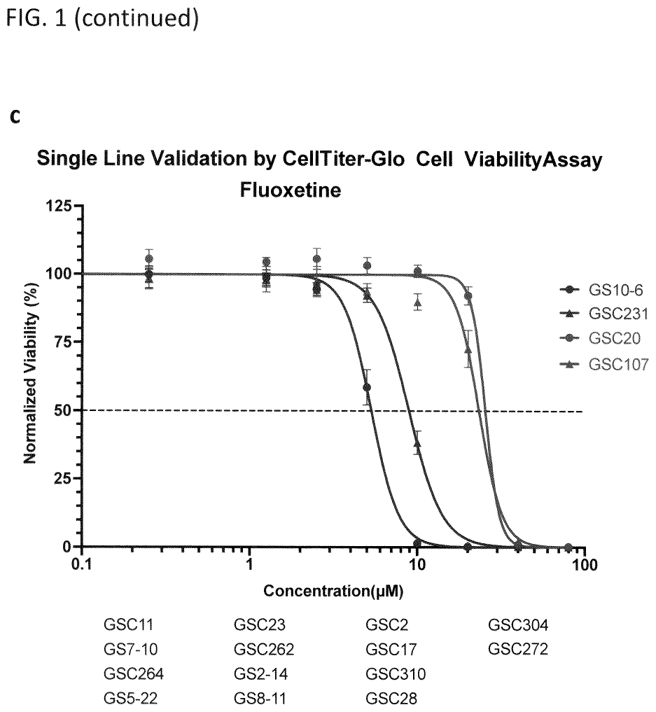

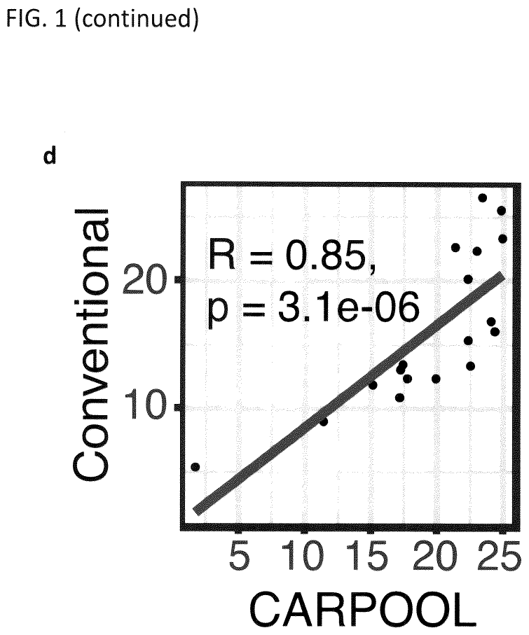

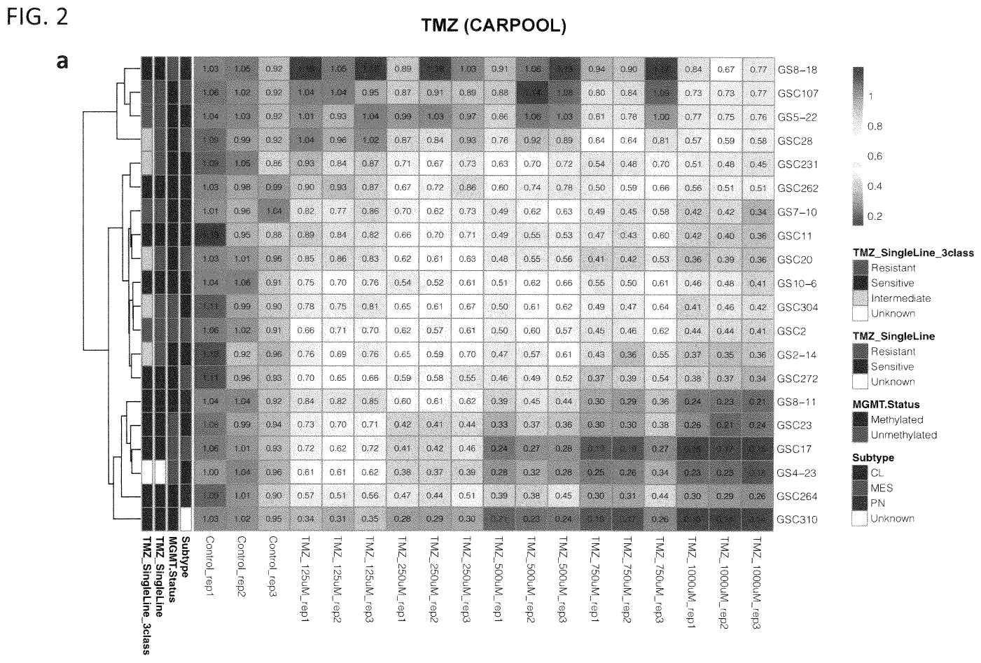

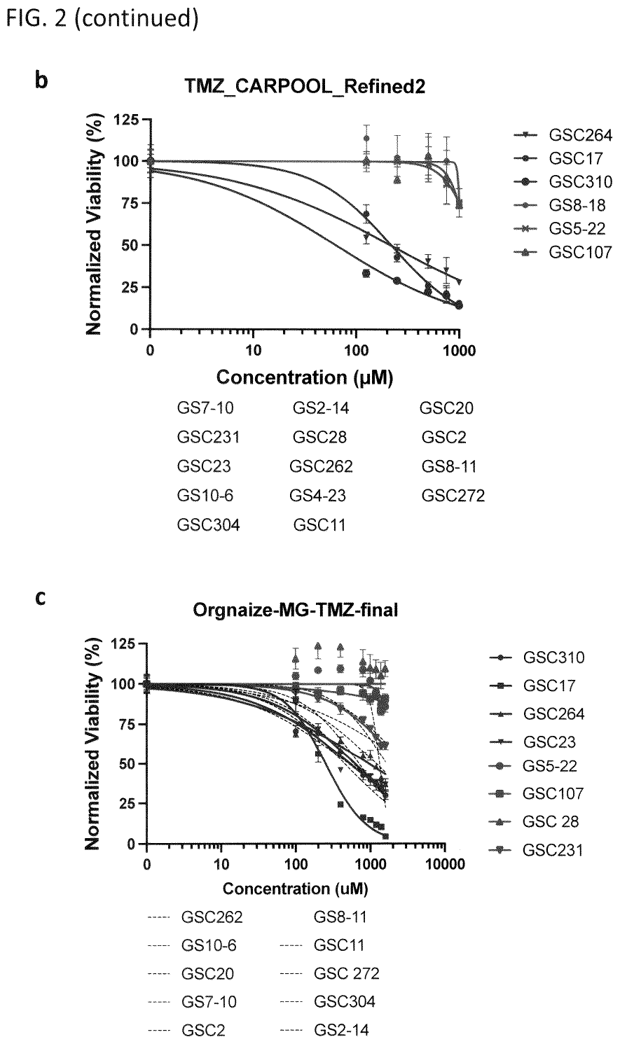

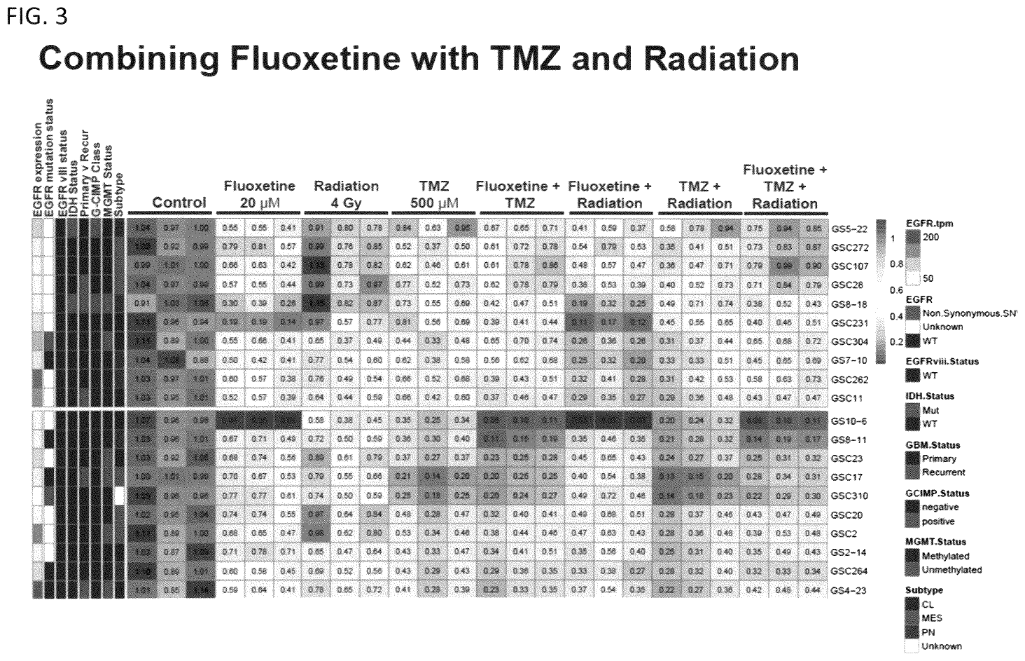

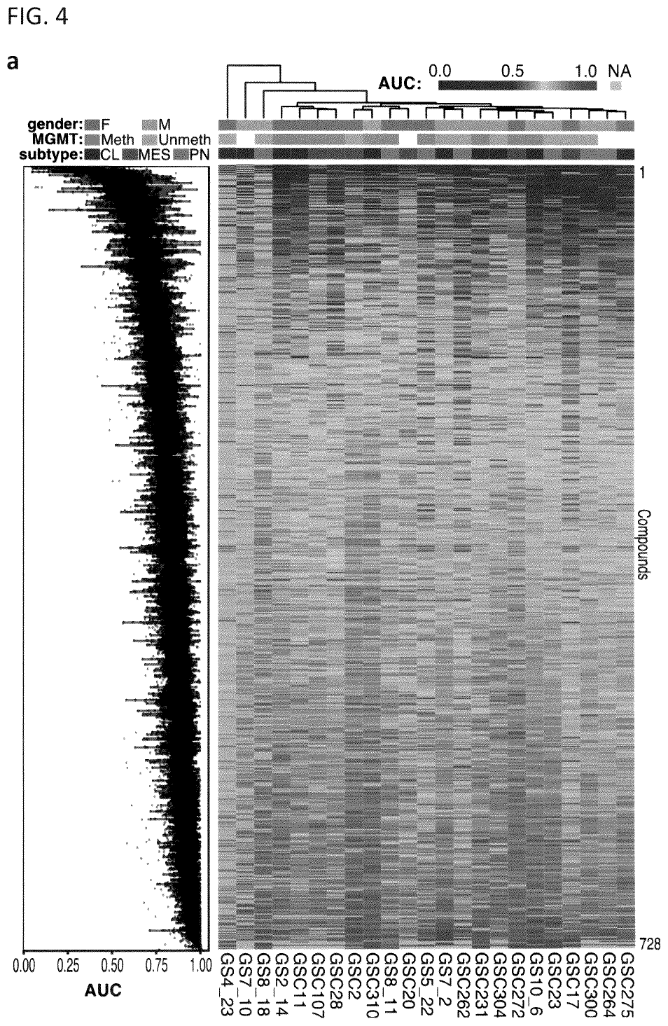

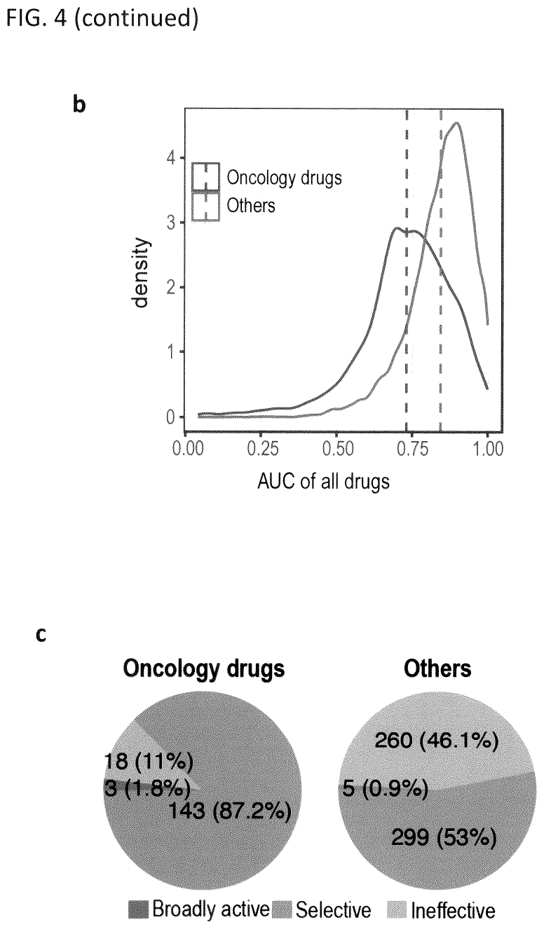

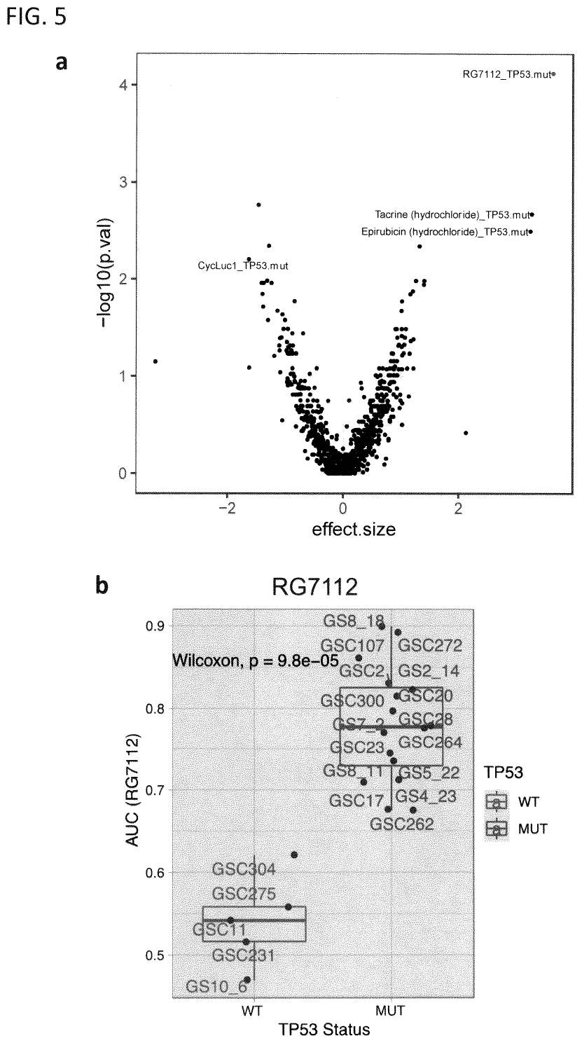

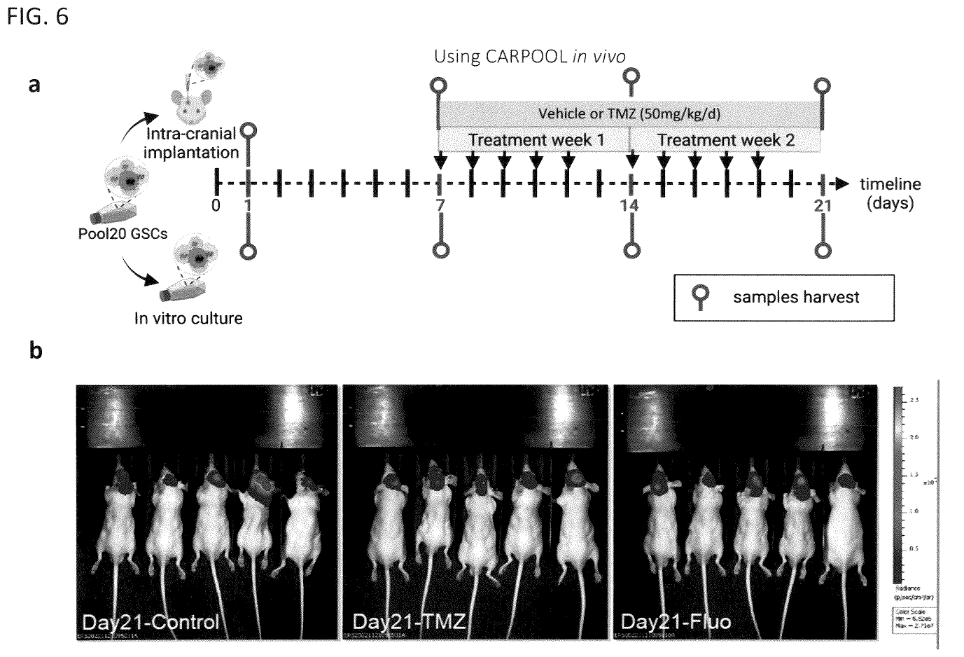

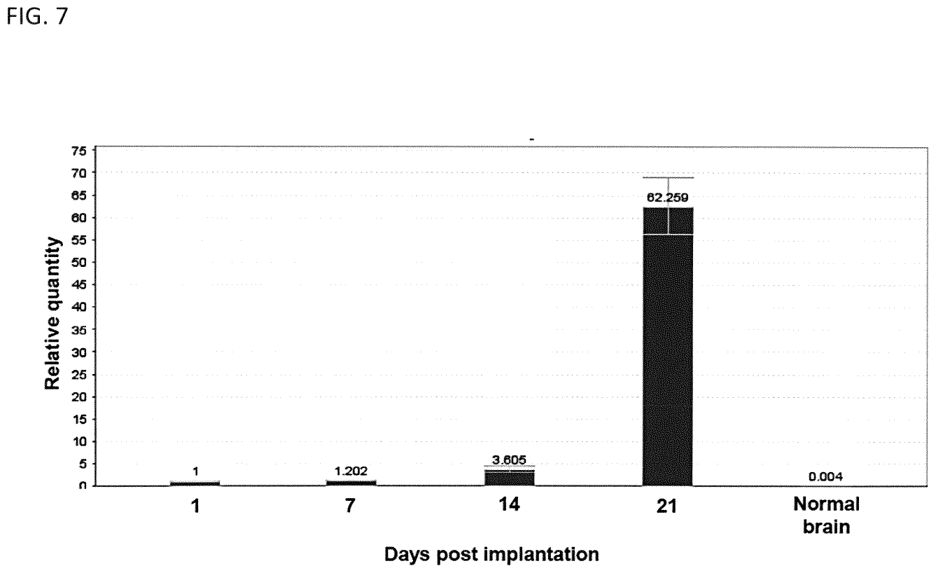

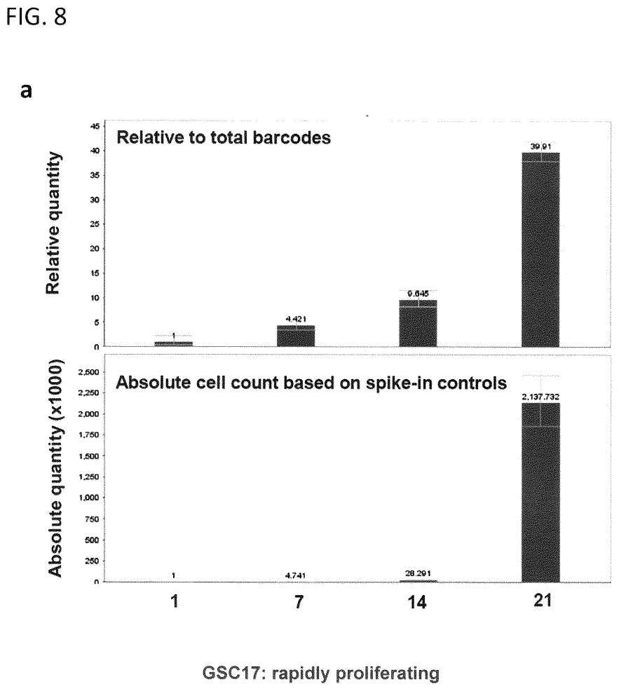

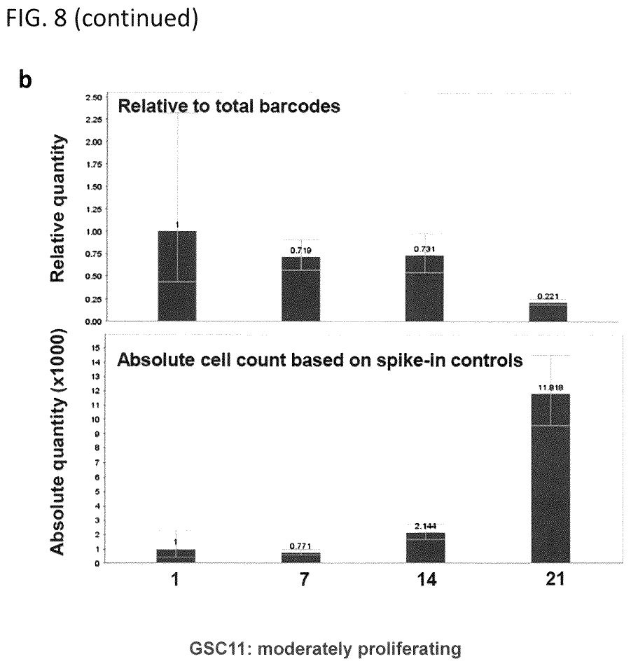

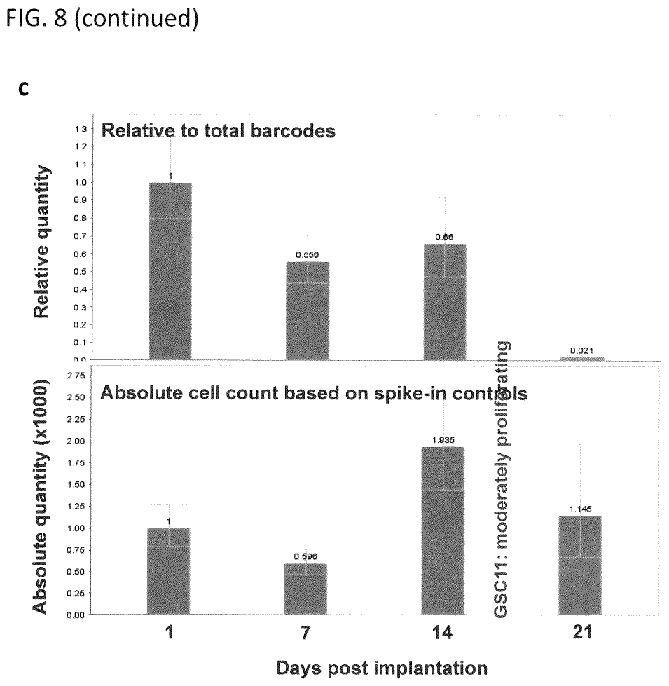

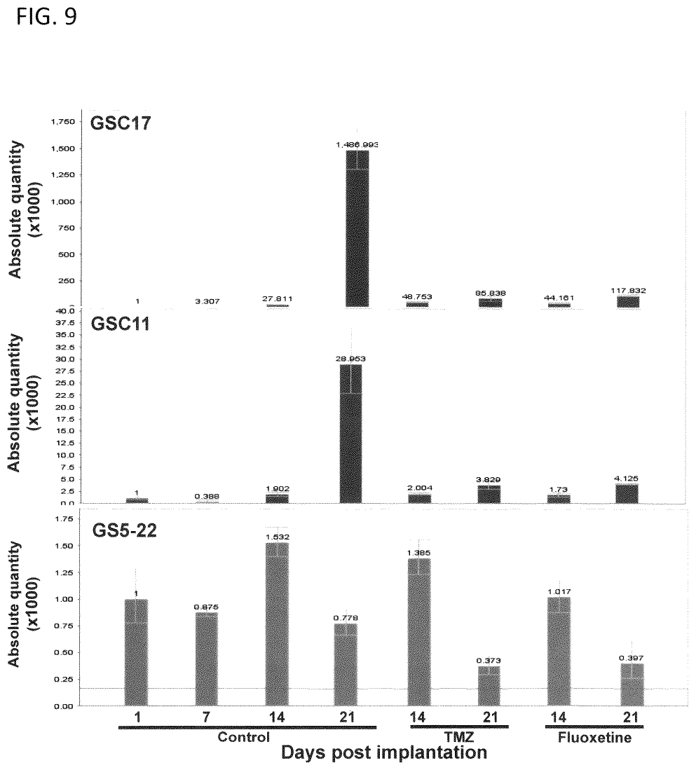

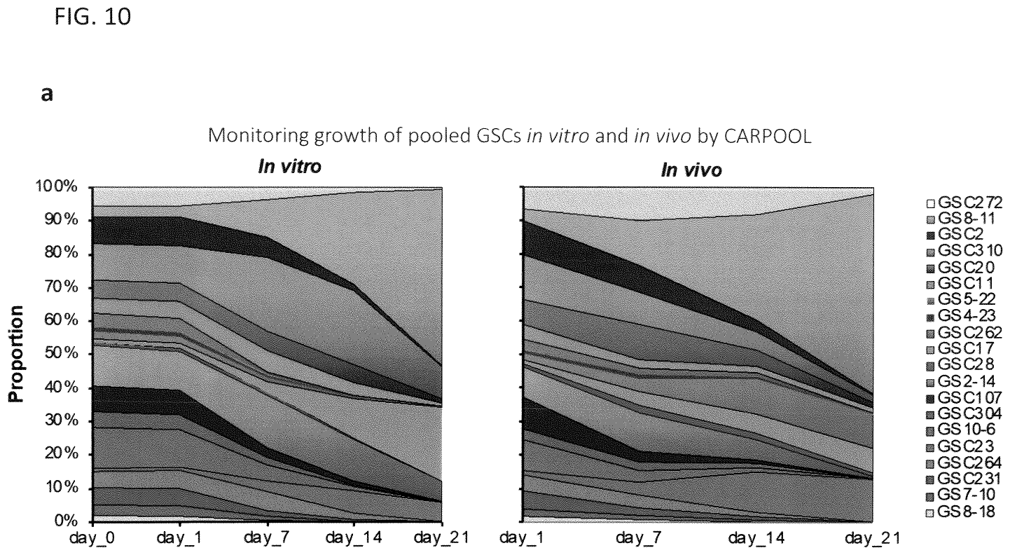

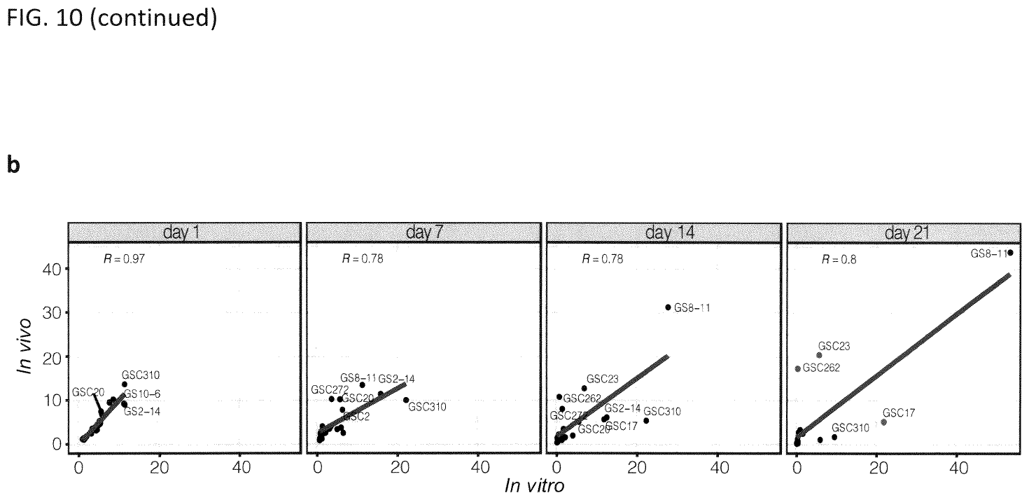

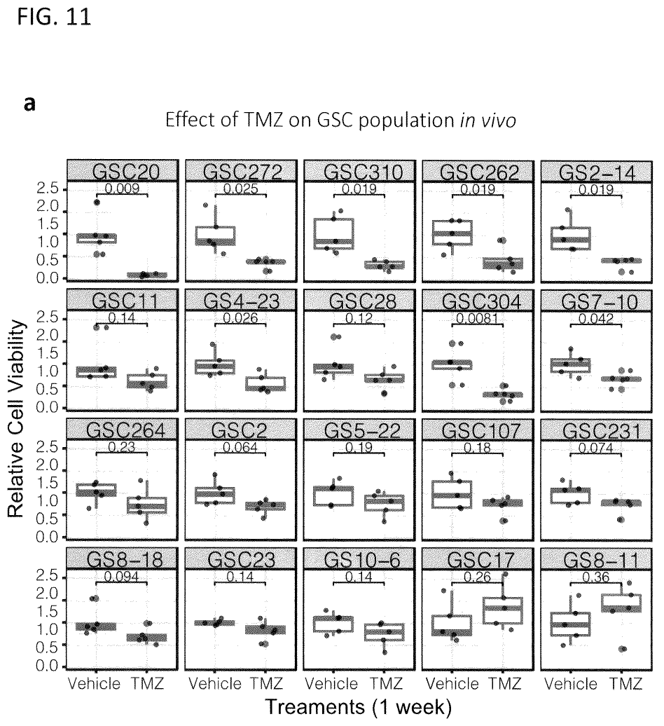

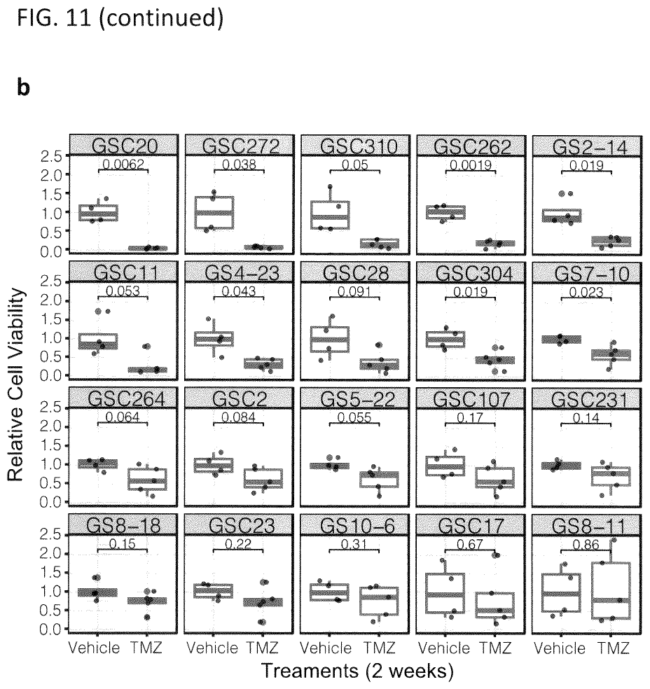

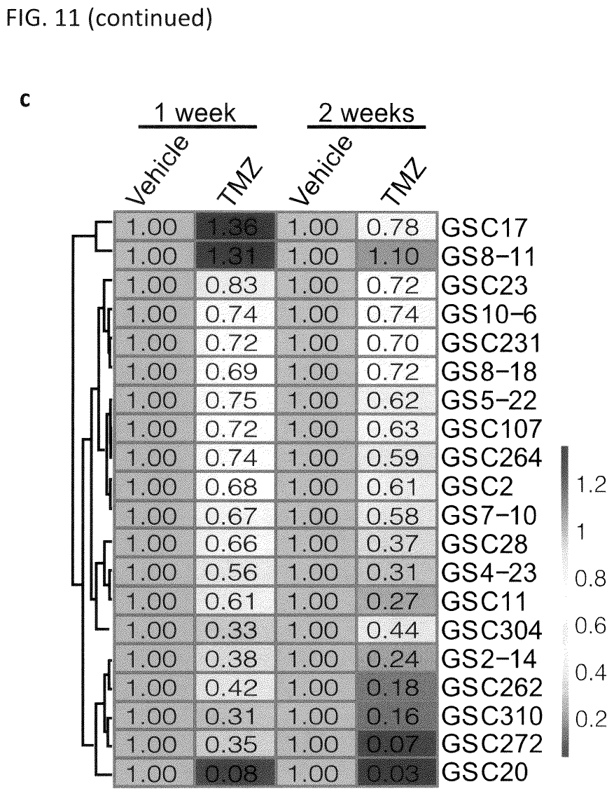

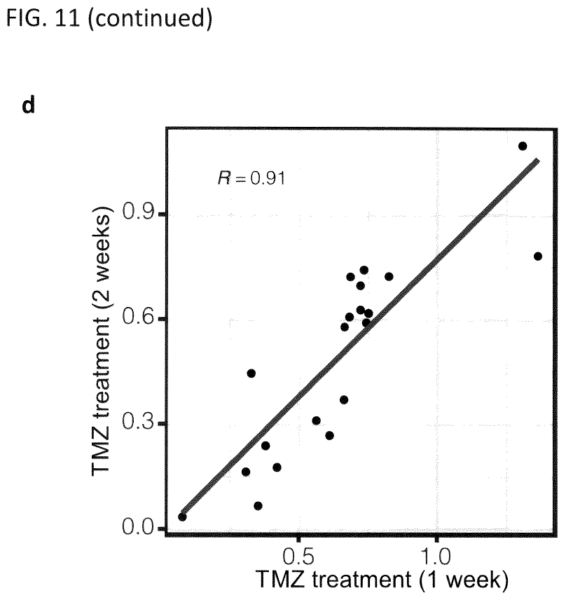

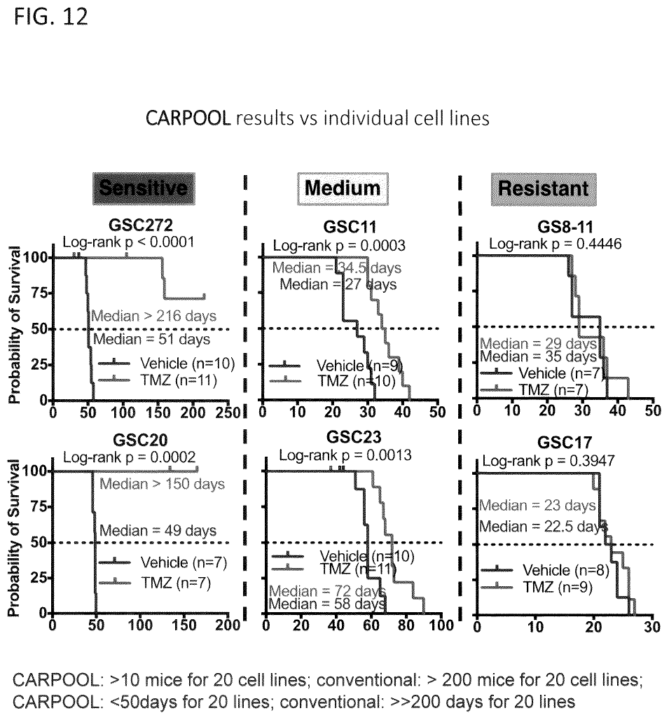

The present disclosure provides compositions and methods referred to herein as “CARPOOL”. CARPOOL is an approach that enables high throughput therapeutic screening in a pool of cell lines which are individually tagged using lentiviral carrying barcodes from CAPTURE library as described in Zhang Z Y, et al., Lineage-coupled clonal capture identifies clonal evolution mechanisms and vulnerabilities of BRAF^{V600E} inhibition resistance in melanoma. Cell Discov. 2022 Oct. 6; 8(1):102. doi: 10.1038/s41421-022-00462-7. PMID: 36202798; PMCID: PMC9537441. (Pubmed ID 36202798), the entire disclosure of which is incorporated herein by reference. A representative barcoding vector that is used in the present disclosure is shown in FIG. 1A of this reference.

In non-limiting examples the disclosure compositions and methods for using CARPOOL in vitro and in vivo.

In examples, the disclosure provides a method comprising:
a) providing a series of groups of test cells; b) barcoding each test cell such that all the test cells in a single group in the series of groups contains the same barcode, and wherein the barcodes are different in each group of test cells; c) pooling the groups of test cells from b) to obtain a pooled combination of the groups of test cells; d) exposing the pooled combination of c) to a test-agent and maintaining the pooled combination in the presence of a test agent for a period of time, and optionally removing dead test cells from the pooled combination of test cells; e) adding a known number of control cells to the pooled combination of d) wherein each of the control cells comprises a control barcode and wherein the control barcode is different from any of the barcodes in the test cells, and wherein control cells are not affected by the test agent; and f) sequencing the barcodes of the test cells and the control barcode of the control cells of e) to determine a difference between the amount of control cells and the amount of test cells to thereby determine an effect of the test agent on each group of the test cells.

In an example, all steps a)-e) described above are performed in vitro. Alternatively, in an example, the pooled combination of the groups of test cells of c) as described above are introduced into a non-human animal and exposed to the test agent within the non-human animal. In this example, the method can further obtain a sample from the non-human animal comprising the test cells after exposure to the test agent, adding a known number of control cells to the sample, and subsequently performing step f) as described above. In examples, the non-human animal from which the test cells are obtained is a rodent, a canine, a feline, an equine, or porcine animal.

The test cells are not particularly limited, and may be any eukaryotic cells. In examples, the test cells are mammalian cells. In examples, the test cells test cells are obtained from a human. In examples, the human from which the test cells is a cancer patient, and thus, test cells may be human patient derived cancer cells. The type of cancer cells are not particularly limited. In non-limiting examples, the cancer cells are of renal cell carcinoma, breast cancer, prostate cancer, pancreatic cancer, lung cancer, liver cancer, ovarian cancer, cervical cancer, colon cancer, esophageal cancer, glioma, glioblastoma or another brain cancer, stomach cancer, bladder cancer, testicular cancer, head and neck cancer, melanoma or another skin cancer, any sarcoma, including but not limited to fibrosarcoma, angiosarcoma, osteosarcoma, and rhabdomyosarcoma, and any blood cancer, including all types of leukemia, lymphoma, and myeloma. In examples, the cancer cells are pancreatic cancer cells, kidney cancer cells, liver cancer cells, or neuroblastoma cells. In embodiments, test cells processed according to this disclosure may be totipotent, pluripotent, oligopotent stem, or multipotent stem cells.

The control cells can be any suitable cells, provided the control cells can be barcoded as described herein, and a known amount of barcoded control cells is used. The control cells may be obtained from the same from the same species as the test cells, and may be of the same tissue type.

The test agent is not particularly limited. “Test agent” as used herein means a compound, small drug molecule, a known or candidate anti-cancer compound including but not limited to a chemotherapeutic drug, a biologic agent, a peptide, or a radiation treatment. In examples, the test agent may be an antibody or antibody derivative, a peptide mimic, a receptor ligand, or a polynucleotide, or a combination thereof. Representative test agents used in this disclosure include but are not limited to temozolomide and fluoxetine.

The disclosure includes isolated groups of test cells that have been modified as described herein. The disclosure includes isolated groups of test cells that have been mixed with a known number of control cells. The disclosure further includes non-human animals that are modified to contain a series of groups of described test cells.

Sequencing of the barcode constructs can be achieved using any suitable technique. In examples, the barcode counts before and after treatment (e.g., exposure to the test agent(s)) are determined by either next-generation sequencing (NGS) or real time, quantitative-PCR (qPCR).

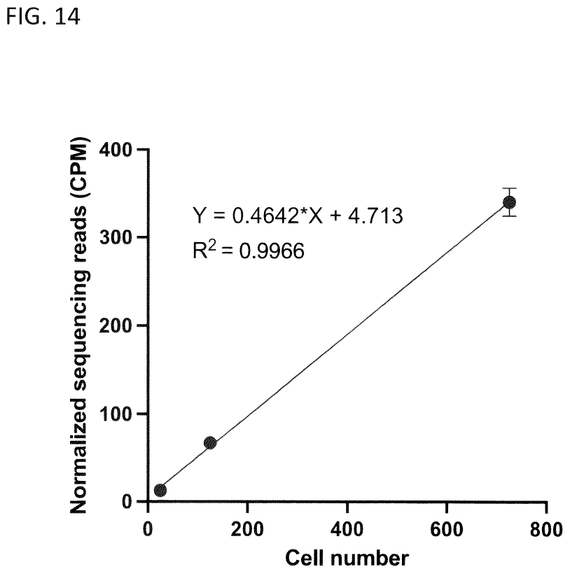

As discussed above, to obtain the absolute count of each cell line in the pool in the control or treatment conditions, cells with additional known barcodes and known counts are spiked in before barcode amplification (spike-in controls, also referred to herein as “control cells”). A standard curve of spike-in cells (cell counts vs. normalized sequencing reads or normalized qPCR expression) is generated and fit with a linear regression. Based on the model, the absolute cell counts for each barcode (cell line) will be deconvoluted. FIG. 14 shows a representative graph.

Non-limiting examples of methods of this disclosure include the following:

An in vivo method comprising:
a) providing a plurality of samples each comprising a plurality of test cells;
b) barcoding each test cell in the plurality of samples with a unique barcode;
c) pooling the plurality of samples from b);
d) introducing pooled cells of c) into a non-human animal;
e) introducing a test agent into the non-human animal of d);
f) extracting the test cells from the non-human animal of e) and adding to the extracted test cells known amount of control cells (spike-in cells) comprising known barcodes, and wherein the test agent does not affect the control cells;
g) extracting genome DNA from cells of f) and preparing next-generation sequencing compatible libraries with indexes for sample analysis;
h) sequencing the DNA libraries of g) to determine the amount of control cells and test cells to thereby determine the effect of the test agent on the test cells.

In addition to h), q-RT-PCR (Quantitative Reverse Transcription PCR) could be used to identify cells of interest by detecting the barcodes to provide an optional quality control step.

Any embodiment of the disclosure may include introduction of sham/vehicle cells as control cells, which are different from the spike-in cells. The spike-in cells are used to generate a cell number standard curve.

In non-limiting examples, the present disclosure provides:

An in vitro method comprising:
a) providing a plurality of samples each comprising a plurality of test cells;
b) barcoding each test cell in the plurality of samples with a unique barcode;
c) pooling the plurality of samples from b);
d) exposing the cells of c) to a test agent and/or a test treatment;
e) adding a known amount of control (spike-in) cells comprising control barcodes, and wherein the test cells are not affected by the test agent;
f) sequencing the DNA of the cells of e) to determine the amount of control cells and test cells to thereby determine the effect of the test agent or the treatment on the test cells.

In an embodiment, the pooled samples of c) are divided into a multiwell assay, and the same or a different test agent is introduced into each well of the multiwell assay.

CARPOOL allows for accurate, parallel interrogation of large numbers of cell lines for a wide variety of applications, include drug sensitivity screening, radiation sensitivity screening, growth condition surveys, and combinatorial evaluations. For example, a typical mouse experiment to study a single cell line might require between 20 and 40 mice. Twenty lines would therefore require 400-800 mice. With the presently described CARPOOL approach, 20 or more cell lines can be evaluated using the same 20-40 mice as single cell line.

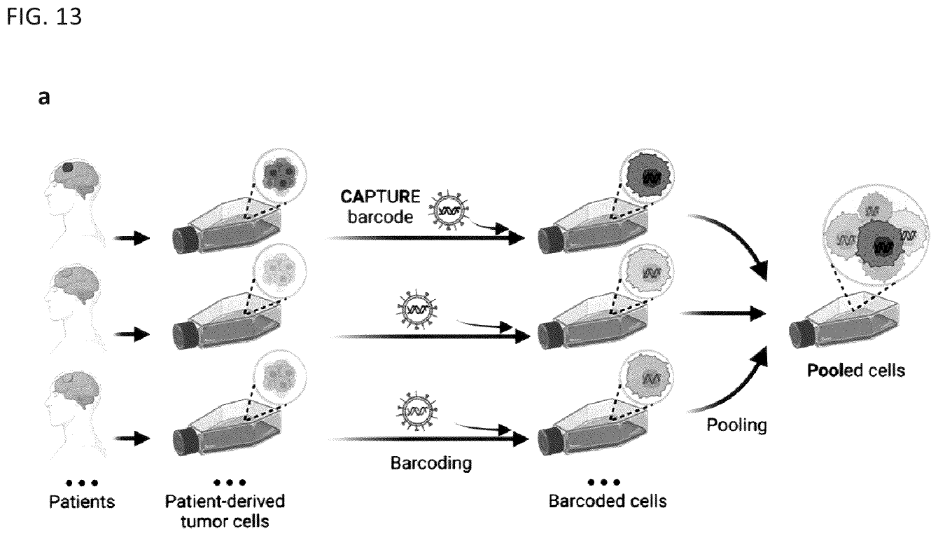

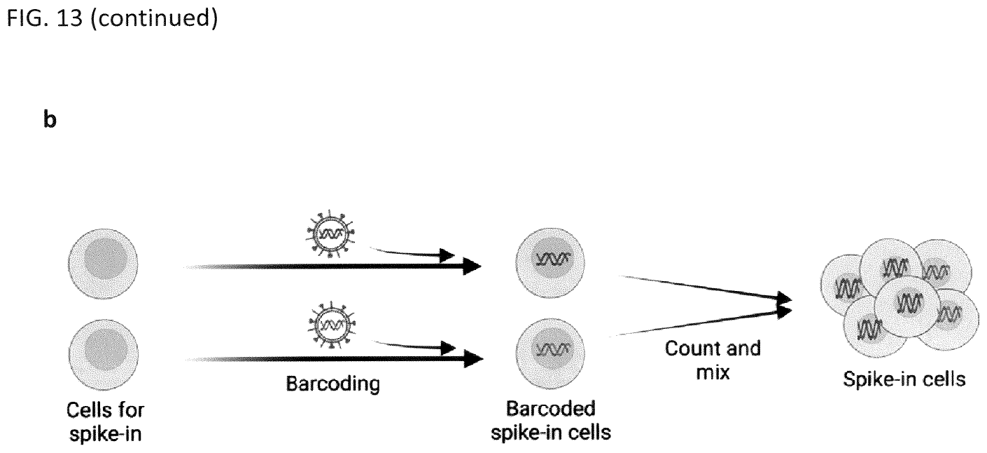

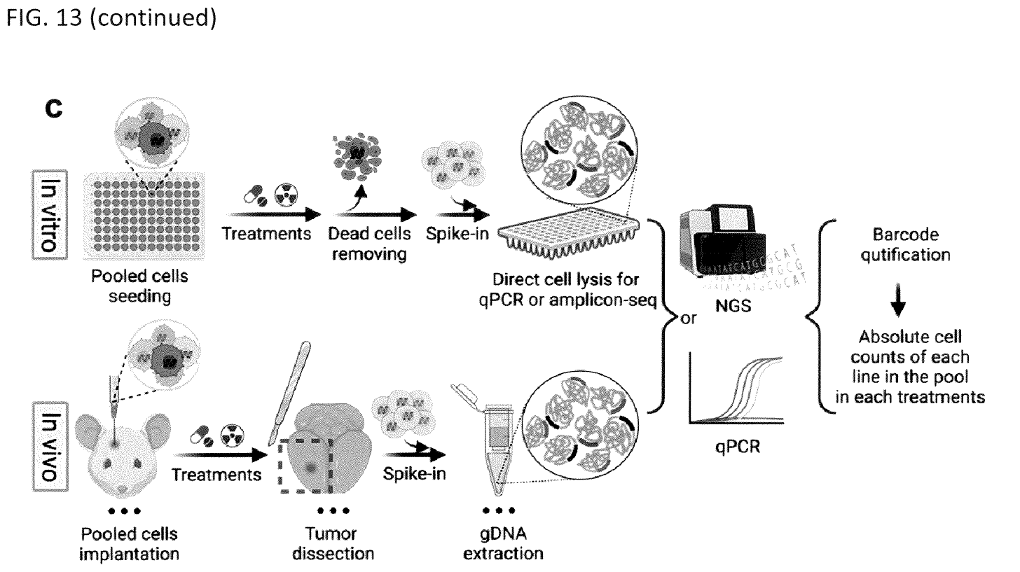

CARPOOL significantly increases the throughput of drug screening by carpooling of multiple lines for treatment. CARPOOL improves the detection sensitivity by obtaining single-cell count resolution. CARPOOL is facile because it is compatible with both regular NGS service or qPCR, although NGS is preferred. A representative and non-limiting illustration of embodiments of the disclosure is provided by the panels of FIG. 13. FIG. 13 panels a b depict the described in vitro approach. FIG. 13 panel c depicts an in vivo approach.

The following description is representative of the materials and methods used to generate the results reflected in the figures that are part of this disclosure.

### Representative Protocol and Materials for Performing CARPOOL

### Regents:

Lentiviral barcodes: Barcodes from The CAPTURE barcode Library as described in the Zhang Z Y, et al. reference
Lentiviral packaging plasmids: psPAX2 and pMD2.G (Addgene)
293T cell
Q5® High-Fidelity 2X Master Mix (NEB, M0492L),
Lysate buffer and DNA additive from Phire Tissue Direct PCR Master Mix (Thermo, F-170L).
Primers
DNA Isolation Kit for Cells and Tissues (Roche, Cat. No. 11814 770 001)
UltraPure Distilled Water (Invitrogen, 10977-015)
COS banker freezing medium (Cosmo bio, COS-CFM01)
PBS (Corning, 21-040-CV)
Select-A-Size DNA clean & Concentrator (Zymo, D4048)
AMPure XP beads (BECKMAN COULTER, A63881)
KAPA Library Quant Kit (Roche, KK4824)
Microseal film (Bio-Rad, MSB1001),
Blasticidin (invivoGen, ant-bi-1)
Polybrene (Sigma, TR-1003-G)

### Equipment:

Thermo Cycler
Nanodrop
qPCR machine (any platform, e.g., ABI ViiA7)
Electrophoresis system for running gels

### High-Throughput Pooling Experiments—Representative Protocol:

### 1. Cells Barcoding and Spike-In Preparation

### A: Barcode-Carrying Lentivirus Packaging:

Co-transfection of lentiviral barcode plasmid (Barcode from CAPTURE) with lentiviral packaging plasmids psPAX2 and pMD2.G at a ratio of 4:3:1 into 293T cells. Wash cells once with PBS and replace serum-containing DMEM for 293T cells to serum-free medium 24 hours after transfection if the target cells are sensitive to serum. For GSCs, change DMEM medium to NBM medium after 24 h of transfection. If the target cells are not sensitive to serum, wash with PBS and change the medium to avoid plasmid carryover in lentiviral preparation. The process includes optionally packaging the lentivirus using dishes that are pre-coated with poly-I-Lysin to facilitate 293T cell attachment. At 48 hours post-transfection, collect the virus-containing medium and add fresh medium. At 72 hours post-transfection, collect the virus-containing medium. Filter the supernatant through a Nalgene 0.45 μm PES filter (a low protein binding filter) to remove debris and floating packaging cells. Aliquot and store the filter lentivirus at −80° C. Freezing and thawing usually results in ^{˜}20% loss of lentiviral titer with each cycle.

### B: Target Cells Barcoding:

Seed cells one night before transduction. Transductions are performed by adding appropriate amount of lentivirus. Polybrene (5 ug/ml) is added if the cells are not sensitive to it. In embodiments different amounts of lentivirus are added to different transduction wells, fluorescent signal is checked 72 hours after transduction to pick the well that MOI <0.3. Barcoded cells are then selected by antibiotics (Blasticidin, usually 5 μg/ml) for about 10 days. The cells are then expanded to produce frozen stocks and for CARPOOL analysis.

### C: Spike-In Preparation:

Spike-ins (i.e., the described control cells) are a series of premixed cells with known number, which do undergo the same therapeutic treatment as the cells of interest (e.g., the control cells are not exposed to the test agent). Spike-ins are added to the endpoint samples after dead-cell removal for in vitro CARPOOL workflow or before genome extraction for in vivo CARPOOL workflow. A cell line (e.g., u87) is transduced using a different set of barcodes separately using the same transduction protocol above. After selection, each of these U87 cells with different barcodes is accurately counted and premixed to serve as spike-ins. The numbers of the spike-ins can range to cover the expected cell number ranges of the barcoded cells of interest, for example ranging from 500% of the average cells number of each barcoded cells to 5% of the average cells number of each barcoded cells. The disclosure encompasses including replicates of spike-ins at each number or at least 5 points for more accurate reads to cell number linear regression standard curve. FIG. 13 is an example spike-in standard curve, from 3 spike-in numbers which each has 3 replicates. Spike-in can be pre-made and aliquoted for later use by keeping in −80° C. In embodiments, spike-ins are prepared at very low MOI (<0.1) so that the cell numbers are closest to barcode numbers.

### 2. Pooling of Barcoded Cells

After barcoding each cell line, the cells are prepared for pooling. In embodiments 20 to 30 cell lines are combined for testing in a 96-well format. On the pooling day, each cell line is enzymatically digested to achieve a single-cell suspension, and the cell count and viability are assessed. For In vitro experiments, the cell lines are mixed in equal proportions based on cell numbers such that each cell line contributes the same percentage to the pool. If the experiments are conducted immediately, the pooled cells are adjusted to the desired cell count and directly seeded into 96-well plates. Alternatively, the pooled cells can be centrifuged and cryopreserved in freezing medium as frozen vials for future use, typically with 10 million cells per vial.

In contrast, for in vivo studies, the mixing of cell lines considers their individual proliferation rates. It is not required to combine cell lines in equal proportions. For instance, in a pool of 20 cell lines, each line may contribute 5% In vitro. However, for in vivo studies, the contribution of a rapidly proliferating line may range from 0.1% to 1%, while a slower growing line may constitute 10%, depending on the experimental requirements.

When utilizing a 96-well plate, in embodiments approximately 50,000 total cells per well are seeded in 100 μl of medium (equivalent to 2,500 cells for each line in a Pool20 setup). To promote preferred representation of each cell line, the disclosure includes using more than 1,000 cells for each line. In embodiments, the disclosure optionally includes pooling no more than 50 cell lines for experiments conducted in a 96-well plate. In embodiments, a preferred range for pooling lines in a 96-well plate experiment is between 20 and 50. Additionally, for in vitro experiments, the use of frozen pool vials is permissible.

### 3. Treatment and Sample Collection

### A: In Vitro:

The assay is adaptable to various treatments, including small molecular compounds, antibodies, peptides, and radiation, as discussed above. Typically, cell treatment (e.g., exposure to the test agent) lasts for 72 hours, but alternative durations are acceptable.

After treatment, for non-adherent cells such as GSCs, an additional step may be introduced to remove dead cells, given that both dead cells and viable cells are in suspension. In an example, before the endpoint of treatment, 10% FBS is added to each well for a period of about 12 hours. This step promotes viable cells attachment to the plate bottom. Conversely, for adherent cell lines, a straightforward and gentle wash with PBS twice is sufficient to remove dead cells at the endpoint of treatment. Following these cell preparation steps, the subsequent procedures for lysate preparation are the same for both non-adherent and adherent cell lines.

### Example of In Vitro Workflow:

(1) On Day 0 (using a Monday morning for illustration), for freshly pooled cells, cells are digested to a single-cell state, and cell count and viability are assessed. Cells are then seeded at 50,000 cells/well in 100 μl of medium in a 96-well plate on Monday afternoon. To mitigate edge evaporation effects, unused wells are surrounded by PBS wells, and cells are cultured overnight. If frozen pool vials are used, the Pool20 stock is revived overnight in advance (Sunday afternoon), digested to a single-cell state, and cell counting is performed the next day (Monday morning).
(2) On Day 1 (Tuesday morning), the corresponding drug/test agent is added to achieve the required final concentration, and the final volume in the 96-well plates is adjusted to 200 μl/well.
(3) On Day 3 (Thursday afternoon, 60 hours), 10% FBS is added to each well (22 μl for a 96-well plate with 200 μl medium) to facilitate the attachment of live GSCs overnight.
(4) On Day 4 (Friday morning, 72 hours), cells are collected. The medium is discarded, wells are washed with PBS once (200 μl/well/96-well plate), and DNA is lysed with dilution buffer (with DNARelease Additive at a 1:40 ratio) following the protocol in Phire Tissue Direct PCR Master Mix (Thermo, F-1701). In brief, 50 μl of dilution buffer is added to each well, and the mixture is incubated at room temperature for 20 minutes. Subsequently, each well is sealed with microseal film (Bio-Rad, MSB1001), and the plate is placed on a preheated block (98° C.) for 10 minutes until the liquid is clear and non-sticky, indicating complete cell lysis. The lysate is then stored at −20° C.

### B: In Vivo

In the in vivo workflow, the dominance of fast-growing cells in the population over weeks of treatment is considered. Consequently, barcoded cells are pooled considering the cell proliferation rate, with fewer fast-growing and more slow-growing cells for optimal results. In embodiments, freshly pooled cells are used on the same day as injection into mice rather than use of frozen pool stocks. Once the cells are pooled, the in vivo experiment can be implemented and treated similarly to a described experiment for a single cell line.

### Example Protocol for GBM Model:

(1) Inject 1×106 GSC pool cells in 5 μl Serum-Free Medium for each mouse, with the cell number in the pool adjusted according to the growth rate.
(2) Treat mice as per described experimental design. For example, administer temozolomide orally daily at 50 mg/kg/mouse for two weeks. On the day of mouse sacrifice, collect the mouse brain, remove unnecessary parts, and retain the region of interest (tumor-burdened area). Weigh the collected brain, wash it once with PBS, and place it in a 1.5 ml EP tube. Typically, each mouse provides around 100-150 mg of brain tissue sample. Store the sample at −80° C.
(3) Add Spike-in Cells to the brain sample before DNA preparation. In this non-limiting example, the Spike-in numbers are 0.25×10⁶/0.05×10⁶/0.01×10⁶ series for 1×10⁶ GSC injection with two weeks of treatment. A suitable DNA Isolation Kit for Cells and Tissues (Roche, Cat. No. 11 814 770 001) is used to process the sample following the manufacturer's instructions. Elute the genomic DNA in water and quantify it using a Nanodrop. For one mouse with 100 mg^{˜}150 mg of brain tissue, typically elute with 500 μl of water, yielding genomic DNA at approximately 100-400 ng/μl. Store the genomic DNA sample at −80° C.
   4. Amplification of Barcodes from Genomic DNA and Purification of Library

When analyzing data through next-generation sequencing (NGS), the choice of primers is adapted to the specific downstream sequencing platform. In non-limiting examples, when using the Illumina HiSeq platform, the disclosure includes using TruSeq-style P7 and P5 primers, as illustrated in the provided table. The design of lentiviral libraries and PCR primers incorporates sequences that are complementary to the immobilized primers used for generating amplification clusters in Illumina's HiSeq Flow Cells. The library design is versatile, accommodating both Single-Read Flow Cells and Paired-End flow cells.

In examples where multiple libraries are pooled together, such as in drug screening experiments, a combination of P5 primers with staggered regions of different lengths is utilized. This strategic design enhances the complexity of the library and reduces signal errors.

For data analysis with qPCR, specific primers containing barcoding sequences are employed. These qPCR-barcode primer designs are compatible with standard qPCR platforms like the ABI ViiA7. The qPCR is executed following the manufacturer's instructions, adhering to the chosen platform specifications.

### A: In Vitro:

### Option 1:

The prepared Spike-in working solution (see representative sequences) Is directly introduced into the PCR system in this step only for in vitro work. For In Vitro work, a sampling range of 10^{˜}20% of the lysated genomic DNA (gDNA) sample is adequate. For instance, in a 96-well plate format, 50 μl per well is lysed, and 5 μl is utilized as the template for each PCR system. Templates do not exceed 8 μl when employing direct lysis buffer, as an excess of lysate buffer can impact PCR efficiency. The PCR conditions are as follows:

| Regent | Final Con. | Volume (μl) |
|---|---|---|
| Q5 2× master Mix | 1× | 25 |
| Primer-F(10 μM) | 0.5 μM | 2.5 |
| Primer-R(10 μM) | 0.5 μM | 2.5 |
| Spike-Ins working solution | | 2 |
| template | | 5 |
| dH20 | | 13 |
| total | | 50 |
| Temp(° C.) | Time(sec) | Cycle |
| 98 | 30 | 1 |
| 98 | 10 | ~30Ct |
| 60 | 20 | |
| 72 | 20-30 | |
| 72 | 10 min | 1 |
| 4 | hold | |

After the PCR step, the library's quality is assessed through gel electrophoresis. In drug screening examples, each well represents a distinct small library with its unique P7 and P5 index. For high-throughput sequencing purposes, a representative 20% sampling from each individual library (e.g., 10 μl from a 50 μl PCR system) is performed. These individual library samples are then combined to create a final library pool for sequencing. The final library pool is subjected to a cleaning process using either AMPure beads or a Select-A-Size DNA cleaning kit, following the manufacturer's protocol. This step removes impurities and enhances the overall quality of the library. Before initiating the sequencing process, the quantification of the library is carried out using the KAPA library quantification kit (Roche, KK4824). This quantification step provides accurate measurements of the library concentration, allowing for precise loading and optimal performance during the subsequent sequencing steps.

### Option 2:

To reduce error introduced during the lysis step, a directly prepared lysis buffer with Spike-in cells pre-mixed can be used.

### 1. Lysis Buffer Preparation:

Add 20 mL of Dilution Buffer (with DNA Release Additive at a 1:40 ratio) to a tube of Sp9-Mix (2M series).
Use 50 μL of homogenized lysis buffer per well in a 96-well plate. Each well will then contain:

| Per 50 ul of Sp9-Mix-InVitro | | |
|---|---|---|
| Series 1 | Series 2 | Series 3 |
| (SpikeIn-1) | (SpikeIn-2) | (SpikeIn-3) |
| 5K R48 | 1K R49 | 200 R50 |
| 5K R30 | 1K R32 | 200 R34 |
| 5K R31 | 1K R33 | 200 R35 |

2. PCR Reaction with 10% Sampling:
Take 5 μL of the dissolved template from each well for PCR.
Each PCR reaction will contain the following SpikeIn amounts:

With 10% sampling = 5 ul of dissolved
template to do PCR, each Reaction contains:

| | | |
|---|---|---|
| 500 R48 | 100 R49 | 20 R50 |
| 500R30 | 100 R32 | 20 R34 |
| 500 R31 | 100 R33 | 20 R35 |

### PCR Reaction is:

| Regent | Final Con. | Volume (μl) |
|---|---|---|
| Q5 2× master Mix | 1× | 25 |
| Primer-F(10 μM) | 0.5 μM | 2.5 |
| Primer-R(10 μM) | 0.5 μM | 2.5 |
| Above Template with Spike-In in it | | 5 |
| dH20 | | 15 |
| total | | 50 |

| Temp(° C.) | Time(sec) | Cycle |
|---|---|---|
| 98 | 30 | 1 |
| 98 | 10 | ~30Ct |
| 60 | 20 | |
| 72 | 20-30 | |
| 72 | 10 min | 1 |
| 4 | hold | |

### 3. Cell Harvesting and Lysis Using Spike-In-Premixed Lysis Buffer:

Use the described process for harvesting cells in the in vitro assay protocol, with replacement of the lysis buffer with Spike-in premixed in the buffer.

On Day 4 (72 hours later), cells are collected. The medium is discarded, wells are washed with PBS once (200 μl/well/96-well plate), and DNA is lysed with dilution buffer (with DNA Release Additive at a 1:40 ratio) following the protocol in Phire Tissue Direct PCR Master Mix (Thermo, F-170L). In brief, 50 μl of dilution buffer is added to each well, and the mixture is incubated at room temperature for 20 minutes. Subsequently, each well is sealed with microseal film (Bio-Rad, MS81001), and the plate is placed on a preheated block (98° C.) for 10 minutes until the liquid is clear and non-sticky, indicating complete cell lysis. The lysate is then stored at −20° C.

### B: In Vivo:

As the Spike-ins has been already incorporated during the DNA preparation step, no additional Spike-in is added during the barcode amplification step for in vivo implementation. Specifically for in vivo examples, a 50% sampling is preferred due to significant distribution differences among barcodes. Sequencing depth should be sufficient to cover low counts adequately.

In this context, the genomic DNA (gDNA) is dissolved in water, enabling the use of a considerable amount of gDNA template in a 50 μl PCR system. Reagent quantities do not adversely affect PCR efficiency. Typically, for brain tissue with a 500 μl gDNA elution (^{˜}100 ng/μl), a 20 μl (2 μg) template is employed. The PCR conditions are as follows:

| Regent | Final Con. | Volume (μl) |
|---|---|---|
| Q5 2× master Mix | 1× | 25 |
| Primer-F(10 μM) | 0.5 μM | 2.5 |
| Primer-R(10 μM) | 0.5 μM | 2.5 |
| template | | 20 |
| total | | 50 |

| Temp(° C.) | Time(sec) | Cycle |
|---|---|---|
| 98 | 30 | 1 |
| 98 | 10 | 30~33Ct |
| 60 | 20 | |
| 72 | 20-30 | |
| 72 | 10 min | 1 |
| 4 | hold | |

To meet the approximately 50% sampling requirement, for one mouse with a 500 μl gDNA elution, 12 tubes of the PCR system are prepared. After assessing the quality of each library by checking the PCR products individually, combine all 12 PCR products together (12×50 μl=600 μl total system volume). Subsequently, clean each library using AMPure beads according to the manufacturer's instructions. It's important to note that each library is prepared individually, and one mouse corresponds to one library.

For the preparation of all libraries, alternative high-fidelity enzymes, such as NEB M0544 or the Phire Animal Tissue Direct PCR Kit (Thermo, F140WH), may also be compatible.

### 5. HT Sequencing of Barcodes

High-throughput sequencing of the pooled amplified barcodes can be conducted on any Illumina sequencing platforms that are compatible with the TruSeq-style sequencing primer, following the manufacturer's protocol. By replacing adapters of the amplicon PCR primers according to other next generation sequencing platforms, the sequencing step are not necessarily limited to the TruSeq-style compatible systems. The required sequencing depth is contingent upon the complexity of the pooled library. The required number of reads can be calculated using the formula:

Reads Needed=Sequencing Depth×Barcode Complexity×Sample Size

### Here,

Sequencing Depth: The desired average sequencing depth (i.e., 1000×).
Barcode Complexity: The diversity or number of unique barcodes in the library.
Sample Size: The number of individual samples in the pooled library.

This formula aids in determining the preferred number of reads to achieve comprehensive coverage and accurate representation of the barcodes within the pooled library during high-throughput sequencing.

### 6. Barcode Enumeration (Conversion of Raw HT Sequencing Data to Number of Reads for Each Barcode)

After sequencing, the barcodes and Spike-in counts are deconvoluted. Subsequently, a linear model is generated using Spike-in's absolute cell numbers and normalized counts. Each barcode then represents the absolute cell number for each cell line, generated based on its normalized counts and the established linear model. The mathematical representation of this process involves the linear model equation:

Absolute Cell Number=(Normalized Counts −Intercept)/Linear Model Coefficient
Absolute Cell Number: The absolute number of cells for a specific cell line.
Normalized Counts: The counts obtained from the sequencing data after normalization.
Linear Model Coefficient: Coefficient derived from the linear model generated using Spike-in's absolute cell numbers.
Intercept: The intercept term from the linear model.

This equation allows for the estimation of the absolute cell number for each cell line based on the normalized counts obtained from the sequencing data and the characteristics of the linear model. For assistance with Barcode Enumeration, technical support can be sought from Zeyan and Yingwen.

### Representatives Sequences

CARPOOL Dual index amplicon (70+198+66=334 bps+0^{˜}8 bp stagger):

(SEQ ID NO: 1)
5′-AATGATACGGCGACCACCGAGATCTACACNNNNNNNNACACTCTTTC
CCTACACGACGCTCTTCCGATCTSgcttccatttcaggtgtcgtgaagcg
gccgcACGCGTccgnnnnnnnnnnnnnnnnnnnngccaccATGgtcgacN
NNNNNNNNNNNNNNNNNNNcggtagcggatccGTGAGCAAGGGCGAGGAG
CTGTTCACCGGGGTGGTGCCCATCCTGGTCGAGCTGGACGGCGACGTAAA
CGGCCACAAGTTCAGCGTGTCCAGATCGGAAGAGCACACGTCTGAACTCC
AGTCACNNNNNNNNATCTCGTATGCCGTCTTCTGCTTG-3′

Primer Table:
Primer for Illumina Hiseq Platform

| | |
|---|---|
| P7 | CAAGCAGAAGACGGCATACGAGATNNNNNNNNGTGACTGGAGTTCA |
| | GACGTGTGCTCTTCCGATCTGGACACGCTGAACTTGTGGC |
| | (SEQ ID NO: 2) |
| P5 | AATGATACGGCGACCACCGAGATCTACACNNNNNNNNACACTCTTT |
| | CCCTACACGACGCTCTTCCGATCT |
| | (SEQ ID NO: 3) |

SpikeIn9 prepare example(numbers in the table
are absolute cell number for each barcode):

| Series 1 | Series 2 | Series 3 | |
|---|---|---|---|
| (SpikeIn-1) | (SpikeIn-2) | (SpikeIn-3) | |
| 4M R48(Sp1-1) | 0.8M R49(Sp2-1) | 0.16M R50 (Sp3-1) | Add 4 ml dilution buffer |
| 4M R30(Sp1-2) | 0.8M R32(Sp2-2) | 0.16M R34(Sp3-2) | with DNARelease Additive |
| 4M R31(Sp1-3) | 0.8M R33(Sp2-3) | 0.16M R35(Sp3-3) | (1:40) (Thermo, F-170L) |

Per 1 μl of SpikeIn 9 - Stock

| | | | |
|---|---|---|---|
| 1K R48 | 200 R49 | 40 R50 | 1:4 dilution, |
| 1K R30 | 200R32 | 40 R34 | Add ddH20 |
| 1K R31 | 200 R33 | 40 R35 | |

Per 1 μl of SpikeIn 9 working solution - diluted (1:4)

| | | | |
|---|---|---|---|
| 250 R48 | 50 R49 | 10 R50 | Each PCR use 2 μl |
| 250 R30 | 50 R32 | 10 R34 | Working solution equals |
| 250 R31 | 50 R33 | 10 R35 | (500 cells/100 cells/ |
| | | | 50 cells |
| | | | standard curve) |

As evidenced by the foregoing description, the CARPOOL system utilizes the CAPTURE lentiviral barcoding vector to label a panel of cell lines with a unique, DNA sequence (or barcode) per line. The lines can then be combined for rapid screening of therapeutics both in vitro and in vivo. CARPOOL includes the vector with a set of barcodes suitable for large number of lines, the technique for barcode insertion, barcoded cell retrieval, DNA sequence library preparation, and the analysis of the sequence to determine sensitive and resistant cell lines in the pool. Thus, CARPOOL is both a set of reagents, a technical guide, and a computational pipeline.

Non-limiting aspects of CARPOOL include the following:
a. Compatible for both in vitro and in vivo settings, enabling high-throughput pre-clinical validation.
b. Next generation sequencing (NGS)-based readout with spike-in normalization allows cell number level sensitivity. qRT-PCR readout provides flexibility, particularly for rapid experiment pre-check before proceeding with NGS.
c. The response of different cell models is evaluated in the same cell culture well or in the same mouse, which minimizes well-to-well or mouse-to-mouse variation compared to previous approaches, thus providing a more reliable cell-to-cell sensitivity comparison.
d. Accelerate the generation of extensive models of therapeutic datasets, expediting AI-driven data discovery. CARPOOL also functions as a high-throughput platform to validate these discoveries.

The disclosure includes the proviso that a described method can be performed without using biotinylated primers, or microbeads that include an antisense barcode, or streptavidin and streptavidin-containing compositions, such as streptavidin-phycoerythrin.

What is claimed is:

1. A method comprising:
a) providing a series of groups of test cells;
b) barcoding each test cell such that all the test cells in a single group in the series of groups contains the same barcode, and wherein the barcodes are different in each group of test cells;
c) pooling the groups of test cells from b) to obtain a pooled combination of the groups of test cells;
d) exposing the pooled combination of c) to a test agent and maintaining the pooled combination in the presence of a test agent for a period of time, and optionally removing dead test cells from the pooled combination of test cells;
e) adding a known number of control cells to the pooled combination of d) wherein each of the control cells comprises a control barcode and wherein the control barcode is different from any of the barcodes in the test cells, and wherein control cells are not affected by the test agent; and
f) sequencing the barcodes of the test cells and the control barcode of the control cells of e) to determine a difference between the amount of control cells and the amount of test cells to thereby determine an effect of the test agent on each group of the test cells.

2. The method of claim 1, wherein steps a)-e) are performed in vitro.

3. The method of claim 1, wherein the pooled combination of the groups of test cells of c) are introduced into a non-human animal and exposed to the test agent within the non-human animal, the method further comprising obtaining a sample from the non-human animal comprising the test cells after exposure to the test agent, adding the known number of control cells to the sample, and subsequently performing step f) of claim 1.

4. The method of claim 1, wherein the test cells are obtained from a human individual before the barcoding.

5. The method of claim 2, wherein the test cells are obtained from a human individual before the barcoding.

6. The method of claim 3, wherein the test cells are obtained from a human individual before the barcoding.

7. The method of claim 6, wherein the test cells are cancer cells.

8. The method of claim 6, wherein the test agent is known or candidate anti-cancer compound, an anti-cancer biologic, a peptide, or a radiation treatment.

9. An isolated series of groups of test cells as in claim 1.

10. The isolated series of groups of test cells of claim 9, each group further comprising a known number of control cells.

11. A non-human animal that is modified to contain a series of groups of test cells as in claim 1.

12. The non-human animal of claim 11, wherein the non-human animal is a mammal.

13. The non-human animal of claim 11, wherein the mammal is a mouse.

14. The non-human animal of claim 13, wherein the groups of test cells are cancer cells.

15. The non-human animal of claim 13, wherein the groups of test cells are human cancer cells.
